(19) United States
(12) Patent Application Publication
(10) Pub. No.: US 2022/0242831 A1
(43) Pub. Date: Aug. 4, 2022

(54) IMPROVED PROCESS FOR THE PREPARATION OF ELAGOLIX AND ITS INTERMEDIATES

(71) Applicant: DR. REDDY'S LABORATORIES LIMITED, Hyderabad (IN)

(72) Inventors: Shiva Kumar KOTA BALAJI, Secunderabad (IN); Rajesh JINKALA, Hyderabad (IN); Venu NALIVELA, Hyderabad (IN); Mohammad JUNAID, Secunderabad (IN); Chandra Shekar Ravi Ram ELATI, Hyderabad (IN); Bhaskara Rao KURETI, Hyderabad (IN); Veerraju ADAPA, Hyderabad (IN); Keshava Rao YERLANKI, Kothagudem (IN); Naga Sailesh SUNKARA, Hyderabad (IN); Sandeep BADARLA, Hyderabad (IN); Vidyadhar SATPUTE, Hyderabad (IN); Nirmal DOLAI, Purba medinipur (IN); Venkatesh CHUKKA, Hyderabad, (IN); Venkatarajesh CHEBOLU, Salur (IN)

(21) Appl. No.: 17/611,804

(22) PCT Filed: May 22, 2020
(86) PCT No.: PCT/IB2020/054884

§ 371 Date: Nov. 16, 2021

(30) Foreign Application Priority Data
May 24, 2019 (IN) 201941020691

(51) Int. Cl. C07D 239/553

(52) U.S. Cl. CPC C07D 239/553

(57) ABSTRACT

The present invention provides an improved process for the preparation of Elagolix sodium of formula (I) and its intermediates. The present invention also provides a compounds of formula (V) and (VI), (X), (Xa) and (Xb). The present invention further provides the use of compounds of formula (V), (VI), (X), (Xa) and (Xb) in the preparation of Elagolix sodium of formula (I). The present invention provides a process for the preparation of Elagolix sodium of formula (I) and its intermediates.

### INTRODUCTION

The present invention provides an improved process for the preparation of Elagolix sodium and its intermediates.

### BACKGROUND

Elagolix sodium is a non-peptide antagonist of the gonadotropin-releasing hormone receptor and chemically known as sodium; 4-[[(1R)-2-[5-(2-fluoro-3-methoxyphenyl)-3-[[2-fluoro-6-(trifluoromethyl)phenyl]methyl]-4-methyl-2,6-dioxopyrimidin-1-yl]-1-phenylethyl]amino]butanoate (I) as given below.

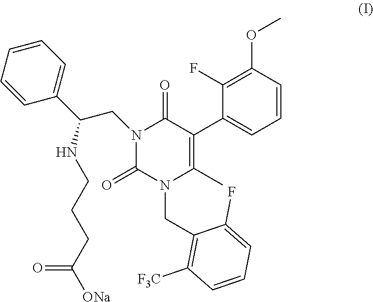

The U.S. Pat. No. 7,056,927 B2 discloses a process for preparation of Elagolix sodium salt in Example-1.

The U.S. Pat. No. 8,765,948 B2 also describes a process for preparation of Elagolix sodium in example-1 and 4.

Further, the U.S. Pat. No. 8,765,948 B2 also discloses an alternate process for the preparation of Elagolix intermediate (formula (1e)).

The present invention provides an improved and commercially viable process for the preparation of Elagolix and its intermediates thereof.

### SUMMARY

First embodiment of the present application provides an improved process for the preparation of Elagolix sodium (I) which includes one or more of the following steps, individually or in the sequence recited:
a) N-protection of compound of formula (IV) to obtain compound of formula (V);

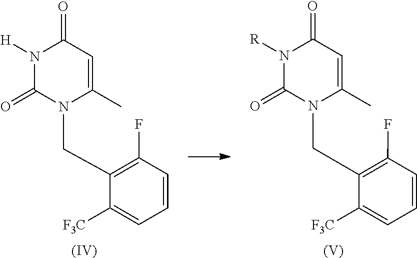

wherein R is para methoxy benzyl, tert-butyloxycarbonyl, methoxymethyl and fluorenylmethyloxycarbonyl.
b) Bromination of compound of formula (V) with a brominating agent to obtain a compound of formula (VI);

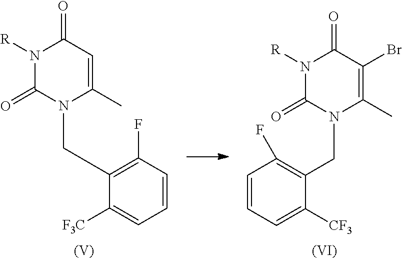

c) reacting a compound of formula (VI) with (2-fluoro-3-methoxyphenyl)boronic acid to obtain a compound of formula (VII);

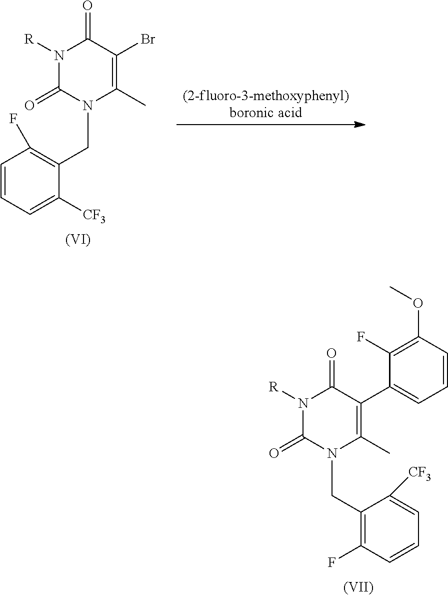

d) reacting a compound of formula (VII) with a reagent to obtain a compound of formula (VIII);

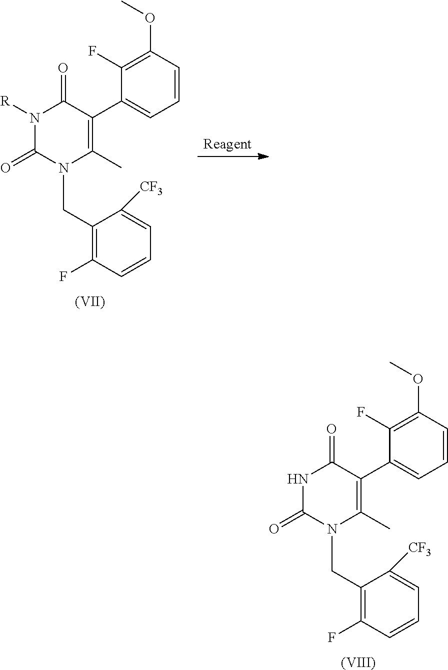

e) reacting a compound of formula (VIII) with (R)-2-((tert-butoxycarbonyl) amino)-2-phenylethyl methanesulfonate (or) tert-butyl (R)-(2-hydroxy-1-phenylethyl) carbamate to obtain a compound of formula (IX);

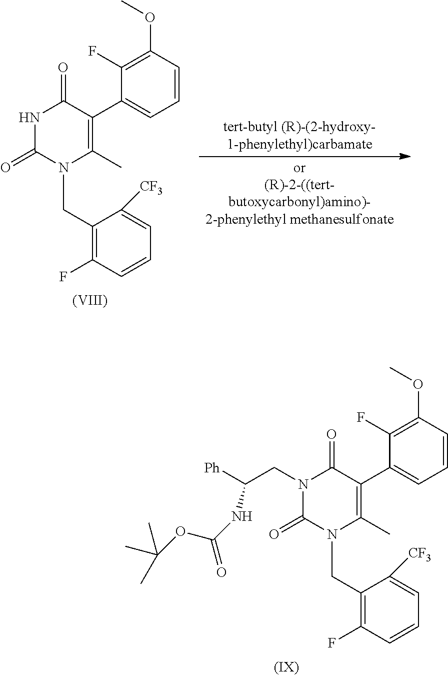

f) converting a compound of formula (IX) in to a compound of formula (XI) or its pharmaceutically acceptable salts;

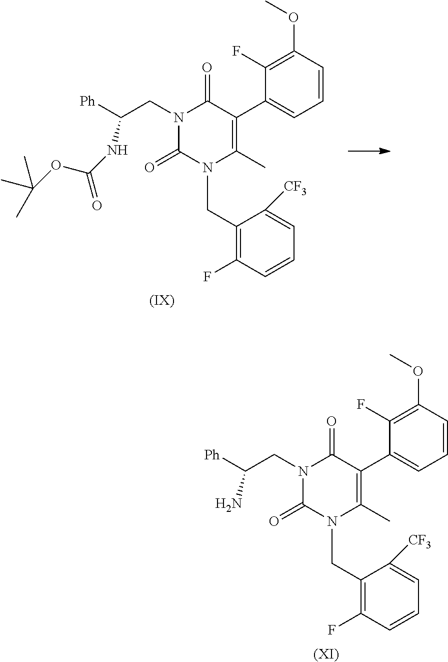

g) reacting a compound of formula (XI) with ethyl 4-bromobutanoate to obtain ethyl ester of Elagolix of formula (XII).

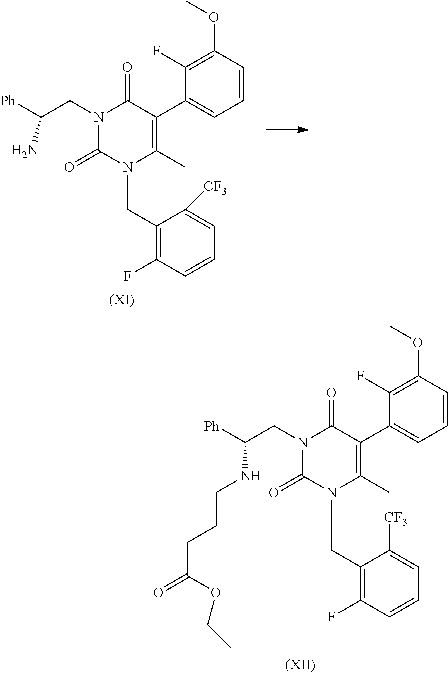

h) converting ethyl ester of Elagolix of formula (XII) into Elagolix sodium of formula (I).

Second embodiment of the present application provides a compounds of formula (V) and (VI);

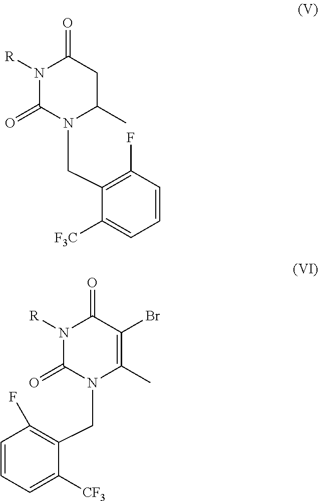

wherein R is para methoxy benzyl, tert-butyloxycarbonyl, methoxymethyl and fluorenylmethyloxycarbonyl.

Third embodiment of the present application provides a compounds of formula (X), (Xa) and (Xb).

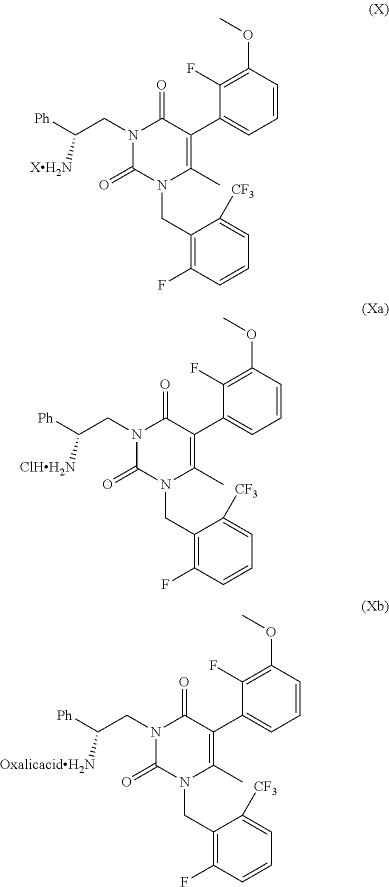

wherein X in compound of formula (X) is hydrochloride, oxalic acid, hydrobromide, tartaric acid, mandelic acid, dibenzoyl-L-tartaric acid and malic acid.

Fourth embodiment of the present application provides the use of compounds of formula (V), (VI), (X), (Xa) and (Xb) in the preparation of Elagolix sodium of formula (I).

Fifth embodiment of the present invention provides a process for the preparation of Elagolix sodium of formula (I) and its intermediates.

### DETAILED DESCRIPTION

First embodiment of the present application provides an improved process for the preparation of Elagolix sodium (I): which includes one or more of the following steps, individually or in the sequence recited:
a) N-protection of compound of formula (IV) to obtain compound of formula (V);

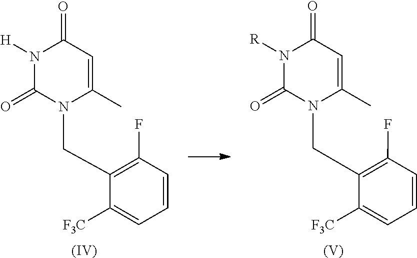

wherein R is para methoxy benzyl, tert-butyloxycarbonyl, methoxymethyl and fluorenylmethyloxycarbonyl.
b) bromination of compound of formula (V) with a brominating agent to obtain a compound of formula (VI);

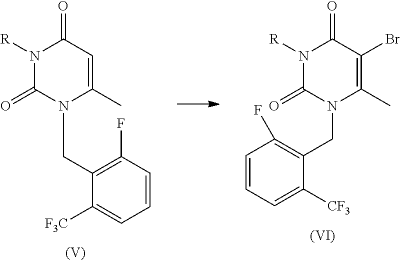

c) reacting a compound of formula (VI) with (2-fluoro-3-methoxyphenyl)boronic acid to obtain a compound of formula (VII);

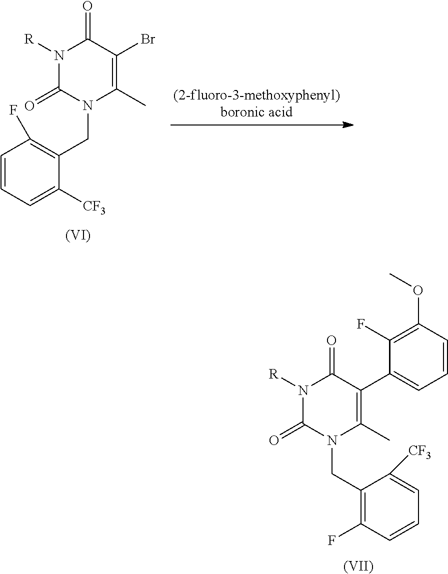

d) reacting a compound of formula (VII) with a reagent to obtain a compound of formula (VIII);

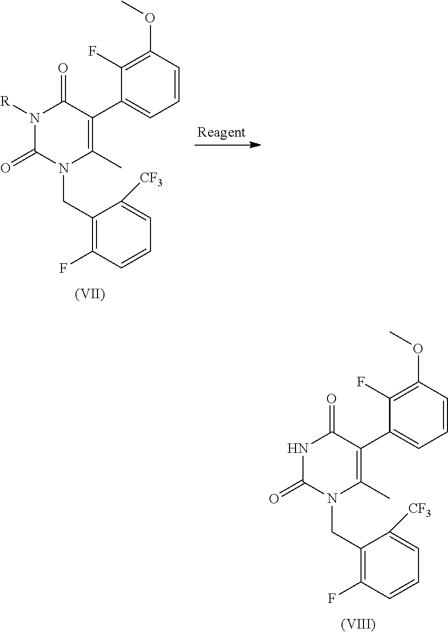

e) reacting a compound of formula (VIII) with (R)-2-((tert-butoxycarbonyl) amino)-2-phenylethyl methanesulfonate (or) tert-butyl (R)-(2-hydroxy-1-phenylethyl)carbamate to obtain a compound of formula (IX);

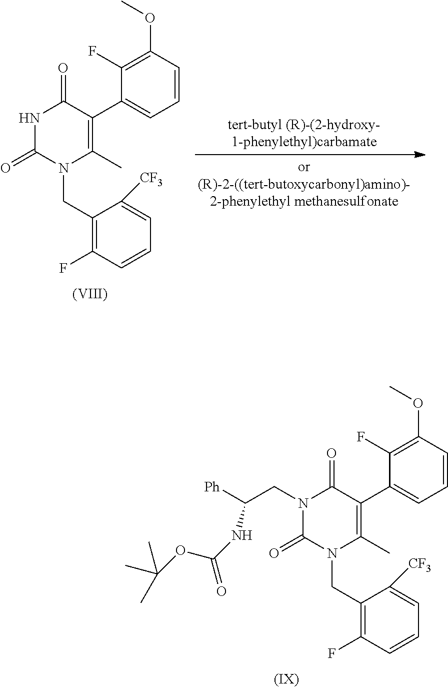

f) converting a compound of formula (IX) in to a compound of formula (XI) or its pharmaceutically acceptable salts;

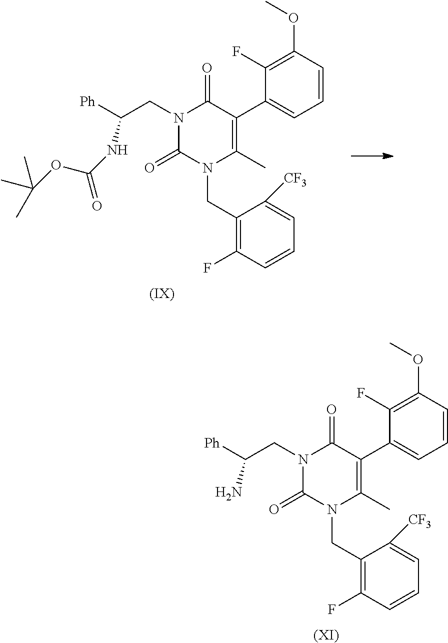

g) reacting a compound of formula (XI) with ethyl 4-bromobutanoate to obtain ethyl ester of Elagolix of formula (XII).

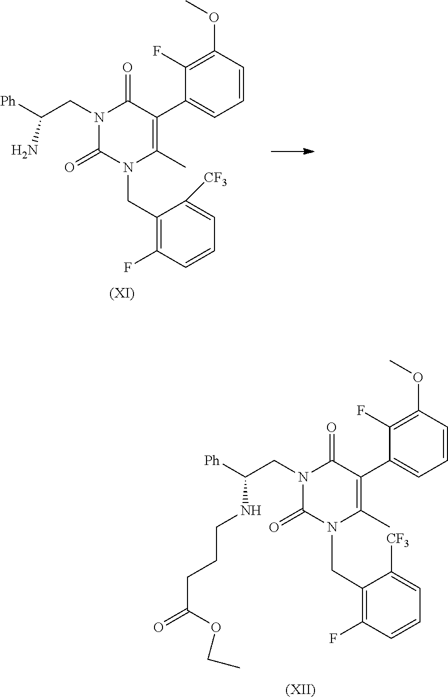

h) converting ethyl ester of Elagolix of formula (XII) into Elagolix sodium of formula (I).

In step a), the compound of formula (IV) is treated with N-protecting group in the presence of a base to give N-protected compound of formula (V). N-protecting groups are selected from para methoxy benzyl, tert-butyloxycarbonyl, methoxymethyl and fluorenyl methyloxycarbonyl and the like.

Suitable solvent may be used in step a) include, but are not limited to polar aprotic solvents such as dimethyl formamide, dimethyl acetamide, N-methyl pyrrolidine, dimethylsulfoxide and the like; halogenated hydrocarbon solvents such as chloroform, dichloromethane, 1,2-dichloroethane, and the like; ether solvents such as tetrahydrofuran, 1,4-dioxane, methyl tertiary butyl ether or mixtures thereof.

Suitable base may be used in step a) include, but are not limited to potassium carbonate, sodium carbonate, lithium carbonate, sodium bicarbonate, potassium bicarbonate, sodium hydride, potassium hydride, and triethylamine, diisopropyl ethyl amine, 1,8-Diazabicyclo[5.4.0]undec-7-ene,1,4-diazabicyclo[2.2.2]octane, 1,1-Dimethylguanidine, Lithium bis(trimethylsilyl)amide, potassium bis(trimethylsilyl)amide and potassium tertbutoxide or mixtures thereof.

Suitable bromination agent may be used in step b) include, but are not limited to phosphorus tribromide, aluminum tribromide, N-bromosuccinimide (NBS) or liquid bromine, N-bromoacetamide, N-bromophthalimide, N-bromosaccharin, benzyltrimethylammoniumTribromide, TrimethylphenylammoniumTribromide, 1,3-Dibromo-5,5-Dimethylhydantoin (DBDMH) and the like.

Suitable solvent may be used in step b) include, but are not limited to water, nitrile solvent such as acetonitrile, propionitrile and the like; ester solvent such as ethyl acetate, propyl acetate, butyl acetate, isobutyl acetate, methyl acetate, ethyl formate and the like; polar aprotic solvents such as dimethyl formamide, dimethyl acetamide, N-methyl pyrrolidine, dimethylsulfoxide and the like; halogenated hydrocarbon solvents such as chloroform, dichloromethane, 1,2-dichloroethane, and the like; aliphatic hydrocarbon solvents such as n-pentane, n-hexane, n-heptane and the like; ether solvents such as tetrahydrofuran, 1,4-dioxane, methyl tertiary butyl ether and mixtures thereof.

Compound of formula (VI) is treated with (2-fluoro-3-methoxyphenyl)boronic acid in presence of ligand, palladium reagent and base to give a compound of formula (VII).

Step c) may be carried out in the presence of palladium reagent such as palladium acetate, palladium(II) chloride, palladium(II) bromide, palladium(II) iodide, palladium(II) nitrate tris(dibenzylideneacetone)dipalladium(O), 1,1′-bis(di-tert-butyl phosphino ferrocene) PdCl2 andtetrakis triphenylphosphine palladium.

Step c) may be carried out in the presence of dialkylbiarylphosphines ligand such as 2-dicyclohexylphosphino-2′,6′-diisopropoxybiphenyl (Ruphos), 2-dicyclohexylphosphino-2′,6′-dimethoxybiphenyl (Sphos), 2-dicyclohexylphosphino-2′-methylbiphenyl, 2-methyl-2′-dicyclohexylphosphinobiphenyl (Mephos), 2-dicyclohexylphosphino-2′-(N,N-dimethylamino)biphenyl (davephos), (2-Biphenyl)di-tert-butylphosphine, (2-Biphenylyl)di-tert-butylphosphine, 2-(Di-tert-butylphosphine) biphenyl (Johnphos), 2-dicyclohexylphosphino-2′,4′,6′-triisopropylbiphenyl (Xphos), 4,5-Bis(diphenylphosphino)-9,9-dimethylxanthene (Xantphos).

Suitable base may be used in step c) include, but are not limited to potassium carbonate, sodium carbonate, cesium carbonate, lithium carbonate, sodium bicarbonate, potassium bicarbonate, potassium hydrogen phosphate, sodium hydroxide, potassium hydroxide, barium hydroxide, potassium fluoride, tetrabutyl ammonium fluoride, triethylamine, tributylamine, N-Methyl-2-pyrrolidone (NMP), N-methylmorpholine, DBU, DABCO and the like or mixtures thereof.

The advantage of using dialkylbiarylphosphines ligands in this step c) can been attributed to a combination of electronic and steric properties that enhances the rates of oxidative addition, trans metalation, and reductive elimination steps in the catalytic cycle of Suzuki reaction. With these ligands it has been shown that oxidative addition of aryl halides is much faster with Pd(0) species than with more highly coordinated complexes.

Suitable solvent may be used in step c) include, but are not limited to ether solvents such as tetrahydrofuran, 1,4-dioxane, methyl tertiary butyl ether and the like; nitrile solvent such as acetonitrile, propionitrile and the like; ester solvent such as ethyl acetate, propyl acetate, butyl acetate, isobutyl acetate, methyl acetate, ethyl formate and the like; alcohol solvents such as methanol, ethanol, isopropyl alcohol, n-butanol, 1-propanol, 2-butanol and the like; polar aprotic solvents such as dimethyl formamide, dimethyl acetamide, N-methyl pyrrolidine, dimethylsulfoxide and the like; halogenated hydrocarbon solvents such as chloroform, dichloromethane, 1,2-dichloroethane, and the like; aromatic hydrocarbon solvents such as toluene, o-xylene, m-xylene, p-xylene and the like; water and mixtures thereof.

Suitable acid may be used in step d) include, but are not limited to aluminium trichlroide, acetic acid, ceric ammonium nitrate, hydrochloride, hydrobromide, trifluoro acetic acid, triflic acid and the like.

Suitable solvent may be used in step d) include, but are not limited to water, ether solvents such as tetrahydrofuran, 1,4-dioxane, anisole, methyl tertiary butyl ether and the like; nitrile solvent such as acetonitrile, propionitrile and the like; ester solvent such as ethyl acetate, propyl acetate, butyl acetate, isobutyl acetate, methyl acetate, ethyl formate and the like; polar aprotic solvents such as dimethyl formamide, dimethyl acetamide, N-methyl pyrrolidine, dimethylsulfoxide and the like; halogenated hydrocarbon solvents such as chloroform, dichloromethane, 1,2-dichloroethane, and the like; aliphatic hydrocarbon solvents such as n-pentane, n-hexane, n-heptane and the like; aromatic hydrocarbon solvents such as toluene, o-xylene, m-xylene, p-xylene and the like; ketone solvents such as acetone, methyl isobutyl ketone and the like or mixtures thereof.

Compound of formula (VIII) is treated with (R)-2-((tert-butoxycarbonyl)amino)-2-phenylethyl methanesulfonate in presence of base to give a compound of formula (IX)

Optionally, compound of formula (VIII) is treated with tert-butyl (R)-(2-hydroxy-1-phenylethyl)carbamate in presence of triphenyl phosphine, di-tert-butyl (E)-diazene-1,2-dicarboxylate to give a compound of formula (IX).

Suitable solvent may be used in step e) include, but are not limited to ether solvents such as tetrahydrofuran, 1,4-dioxane, anisole, methyl tertiary butyl ether and the like; nitrile solvent such as acetonitrile, propionitrile and the like; ester solvent such as ethyl acetate, propyl acetate, butyl acetate, isobutyl acetate, methyl acetate, ethyl formate and the like; polar aprotic solvents such as dimethyl formamide, dimethyl acetamide, N-methyl pyrrolidine, dimethylsulfoxide and the like; halogenated hydrocarbon solvents such as chloroform, dichloromethane, 1,2-dichloroethane, and the like; aliphatic hydrocarbon solvents such as n-pentane, n-hexane, n-heptane and the like; aromatic hydrocarbon solvents such as toluene, o-xylene, m-xylene, p-xylene and thereof.

Suitable base may be used in step e) include, but are not limited to potassium carbonate, sodium carbonate, cesium carbonate, lithium carbonate, sodium bicarbonate, potassium bicarbonate, potassium hydrogen phosphate, sodium hydroxide, potassium hydroxide, barium hydroxide, potassium fluoride, tetrabutyl ammonium fluoride, triethylamine, tributylamine, N-Methyl-2-pyrrolidone (NMP), N-methylmorpholine, DBU, DABCO and the like or mixtures thereof.

Compound of formula (IX) is treated with an acid to give a compound of formula (XI).

Suitable acid that may be used in step f) include, but are not limited to hydrochloric acid, sulphuric acid, hydrobromic acid, acetic acid, orthophosphoric acid, trifluoroacetic acid, methane sulfonic acid and the like or combinations thereof.

Suitable solvent may be used in step f) include, but are not limited to ether solvents such as tetrahydrofuran, 1,4-dioxane, anisole, methyl tertiary butyl ether and the like; nitrile solvent such as acetonitrile, propionitrile and the like; ester solvent such as ethyl acetate, propyl acetate, butyl acetate, isopropyl acetate, isobutyl acetate, methyl acetate, ethyl formate and the like; alcohol solvents such as methanol, ethanol, isopropyl alcohol, n-butanol, 1-propanol, 2-butanol and the like; polar aprotic solvents such as, dimethylsulfoxide and the like; halogenated hydrocarbon solvents such as chloroform, dichloromethane, 1,2-dichloroethane, and the like; water and mixtures thereof.

Compound of formula (XI) is treated with salt source to give a compound of formula (X). wherein salt source is selected from hydrochloride, oxalic acid, hydrobromide, tartaric acid, mandelic acid, dibenzoyl-L-tartaric acid and malic acid.

Compound of formula (XI) is treated with ethyl 4-bromobutanoate in presence of a base to produce ethyl ester of elagolix of formula (XII).

Suitable base that may be used in step g) include, but are not limited to pyridine, piperidine, pyrimidine, triethylamine, tributylamine, N-methylmorpholine, N,N-diisopropylethylamine, diethylamine, DBU, DABCO and the like.

Ethyl ester of elagolix of formula (XII) is treated with sodium source in presence of solvent to produce Elagolix sodium of formula (I).

Suitable solvent that may be used in step g) include, but are not limited to ether solvents such as tetrahydrofuran, 1,4-dioxane, anisole, methyl tertiary butyl ether and the like; nitrile solvent such as acetonitrile, propionitrile and the like; alcohol solvents such as methanol, ethanol, isopropyl alcohol, n-butanol, 1-propanol, 2-butanol and the like; polar aprotic solvents such as dimethyl formamide, dimethyl acetamide, N-methyl pyrrolidine, dimethylsulfoxide and the like; halogenated hydrocarbon solvents such as chloroform, dichloromethane, 1,2-dichloroethane, and the like; ester solvent such as ethyl acetate, propyl acetate, t-butyl acetate, isopropyl acetate, isobutyl acetate, methyl acetate, ethyl formate and the like, water, orthophosporic acid and mixtures thereof.

The temperature at which the above steps may be carried out in between about −30° C. and about 200° C., preferably at about 0° C. and about 150° C., most preferably at about −10° C. and about 100° C., based on the solvent or mixture of solvent used in particular step.

The intermediates obtained in the present application may be directly used for the next step with or without isolation or it may be further purified, if isolated, to improve the purity of the product.

The isolation of Elagolix sodium of formula (I) may be effected, if desired, by any suitable separation or purification procedure such as, for example, filtration, centrifugation, extraction, acid-base treatment, crystallization, conventional isolation and refining means such as concentration, concentration under reduced pressure, solvent-extraction, crystallization, phase-transfer chromatography, column chromatography, or by a combination of these procedures.

Second embodiment of the present application provides a compounds of formula (V) and (VI);

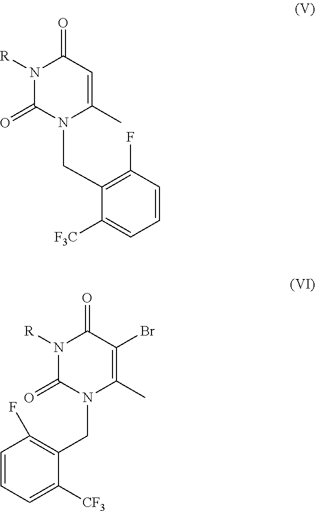

wherein R is para methoxy benzyl, tert-butyloxycarbonyl, methoxymethyl and fluorenylmethyloxycarbonyl.

Third embodiment of the present application provides a compounds of formula (X), (Xa) and (Xb).

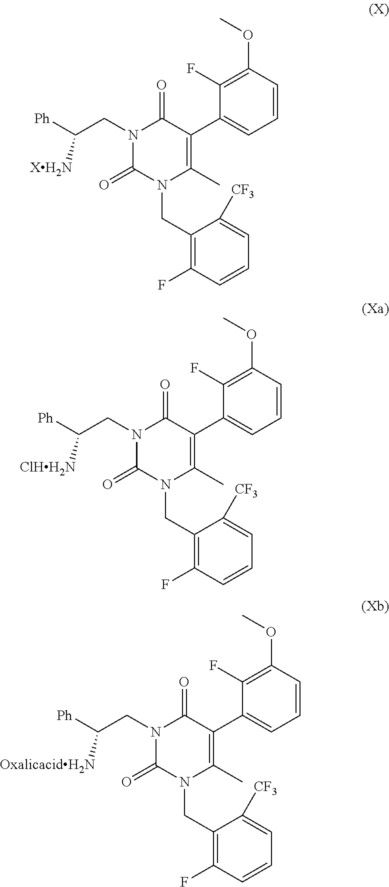

wherein X in compound of formula (X) is hydrochloride, oxalic acid, hydrobromide, tartaric acid, mandelic acid, dibenzoyl-L-tartaric acid and malic acid.

Fourth embodiment of the present application provides the use of compounds of formula (V), (VI), (X), (Xa) and (Xb) in the preparation of Elagolix sodium of formula (I).

Fifth embodiment of the present application provides a process for the preparation of Elagolix sodium of formula (I) which comprises:
a) reacting a compound of formula (XIII) with (2-fluoro-3-methoxyphenyl)boronic acid to obtain a compound of formula (IX);

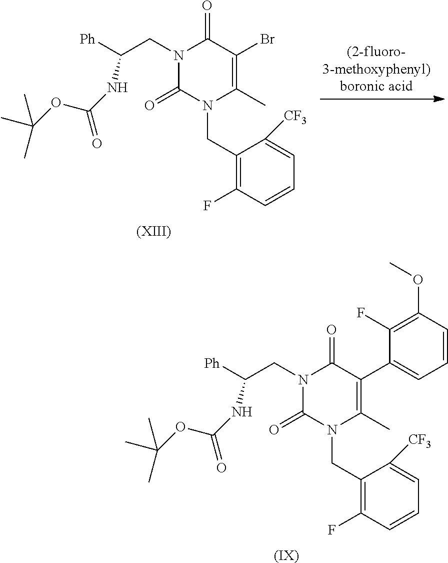

b) converting a compound of formula (IX) to Elagolix sodium of formula (I)

Compound of formula (XIII) is treated with (2-fluoro-3-methoxyphenyl)boronic acid in presence of ligand, palladium reagent and base to give a compound of formula (IX).

Step a) may be carried out in the presence of palladium reagent such as palladium acetate, palladium(II) chloride, palladium(II) bromide, palladium(II) iodide, palladium(II) nitrate tris(dibenzylideneacetone)dipalladium(O), 1,1′-bis(di-tert-butyl phosphino ferrocene) PdCl2 andtetrakis triphenylphosphine palladium.

Step a) may be carried out in the presence of dialkylbiarylphosphines ligands such as 2-dicyclohexylphosphino-2′,6′-diisopropoxybiphenyl (Ruphos), 2-dicyclohexylphosphino-2′,6′-dimethoxybiphenyl (Sphos), 2-dicyclohexylphosphino-2′-methylbiphenyl, 2-methyl-2′-dicyclohexylphosphinobiphenyl (Mephos), 2-dicyclohexylphosphino-2′-(N,N-dimethylamino)biphenyl (davephos), (2-Biphenyl)di-tert-butylphosphine, (2-Biphenylyl)di-tert-butylphosphine, 2-(Di-tert-butylphosphine) biphenyl (Johnphos), 2-dicyclohexylphosphino-2′,4′,6′-triisopropylbiphenyl (Xphos), 4,5-Bis(diphenylphosphino)-9,9-dimethylxanthene (Xantphos).

Suitable base may be used in step a) include, but are not limited to Suitable solvent may be used in step a) include, but are not limited to ether solvents such as tetrahydrofuran, 1,4-dioxane, methyl tertiary butyl ether and the like; nitrile solvent such as acetonitrile, propionitrile and the like; ester solvent such as ethyl acetate, propyl acetate, butyl acetate, isobutyl acetate, methyl acetate, ethyl formate and the like; alcohol solvents such as methanol, ethanol, isopropyl alcohol, n-butanol, 1-propanol, 2-butanol and the like; polar aprotic solvents such as dimethyl formamide, dimethyl acetamide, N-methyl pyrrolidine, dimethylsulfoxide and the like; halogenated hydrocarbon solvents such as chloroform, dichloromethane, 1,2-dichloroethane, and the like; aromatic hydrocarbon solvents such as toluene, o-xylene, m-xylene, p-xylene and the like; water and mixtures thereof.

Converting a compound of formula (IX) into Elagolix sodium of formula (I) by the processes known in the literature.

The temperature at which the above steps may be carried out in between about −30° C. and about 200° C., preferably at about 0° C. and about 150° C., most preferably at about 0° C. and about 100° C., based on the solvent or mixture of solvent used in particular step.

The intermediates obtained in the present application may be directly used for the next step with or without isolation or it may be further purified, if isolated, to improve the purity of the product.

Sixth embodiment of the present application provides a process for the preparation of Elagolix sodium of formula (I) which comprises:
a) reacting a compound of formula (XIV) with (2-fluoro-3-methoxyphenyl) boronic acid to obtain a compound of formula (VIII);

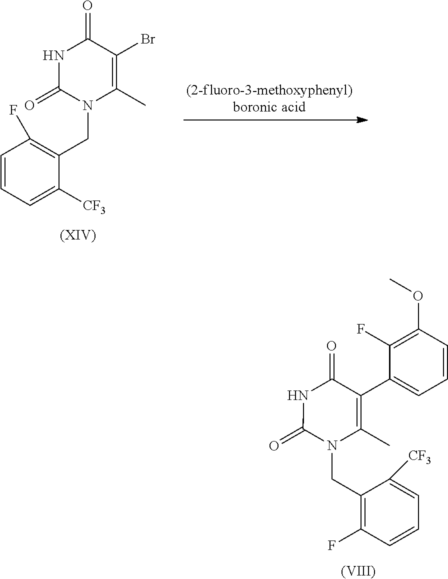

b) converting a compound of formula (IX) to Elagolix sodium of formula (I).

Compound of formula (XIV) is treated with (2-fluoro-3-methoxyphenyl)boronic acid in presence of ligand, palladium reagent and base to give a compound of formula (VIII).

Step a) may be carried out in the presence of palladium reagent such as palladium acetate, palladium(II) chloride, palladium(II) bromide, palladium(II) iodide, palladium(II) nitrate tris(dibenzylideneacetone)dipalladium(O), 1,1′-bis(di-tert-butyl phosphino ferrocene) PdCl2 andtetrakis triphenylphosphine palladium.

Step a) may be carried out in the presence of dialkylbiarylphosphines ligands such as 2-dicyclohexylphosphino-2′,6′-diisopropoxybiphenyl (Ruphos), 2-dicyclohexylphosphino-2′,6′-dimethoxybiphenyl (Sphos), 2-dicyclohexylphosphino-2′-methylbiphenyl, 2-methyl-2′-dicyclohexylphosphinobiphenyl (Mephos), 2-dicyclohexylphosphino-2′-(N,N-dimethylamino)biphenyl (davephos), (2-Biphenyl)di-tert-butylphosphine, (2-Biphenylyl)di-tert-butylphosphine, 2-(Di-tert-butylphosphine) biphenyl (Johnphos), 2-dicyclohexylphosphino-2′,4′,6′-triisopropylbiphenyl (Xphos), 4,5-Bis(diphenylphosphino)-9,9-dimethylxanthene (Xantphos).

Suitable base may be used in step a) include, but are not limited to potassium carbonate, sodium carbonate, cesium carbonate, lithium carbonate, sodium bicarbonate, potassium bicarbonate, potassium hydrogen phosphate, sodium hydroxide, potassium hydroxide, barium hydroxide, potassium fluoride, tetrabutyl ammonium fluoride, triethylamine, tributylamine, N-Methyl-2-pyrrolidone (NMP), N-methylmorpholine, DBU, DABCO and the like or mixtures thereof.

Suitable solvent may be used in step a) include, but are not limited to ether solvents such as tetrahydrofuran, 1,4-dioxane, methyl tertiary butyl ether and the like; nitrile solvent such as acetonitrile, propionitrile and the like; ester solvent such as ethyl acetate, propyl acetate, butyl acetate, isobutyl acetate, methyl acetate, ethyl formate and the like; alcohol solvents such as methanol, ethanol, isopropyl alcohol, n-butanol, 1-propanol, 2-butanol and the like; polar aprotic solvents such as dimethyl formamide, dimethyl acetamide, N-methyl pyrrolidine, dimethylsulfoxide and the like; halogenated hydrocarbon solvents such as chloroform, dichloromethane, 1,2-dichloroethane, and the like; aromatic hydrocarbon solvents such as toluene, o-xylene, m-xylene, p-xylene and the like; water and mixtures thereof.

Converting a compound of formula (VIII) into Elagolix sodium of formula (I) by the processes known in the literature.

The intermediates obtained in the present application may be directly used for the next step with or without isolation or it may be further purified, if isolated, to improve the purity of the product.

In each stage the compounds of all embodiments of the present application are isolated from the reaction mixture may involve methods including removal of solvent, cooling, crash cooling, concentrating the mass, evaporation, flash evaporation, simple evaporation, fast solvent evaporation, rotational drying, spray drying, thin-film drying, agitated thin film drying, agitated nutsche filter drying, pressure nutsche filter drying, freeze-drying, rotary vacuum paddle dryer, adding anti-solvent or the like. Stirring or other alternate methods such as shaking, agitation, or the like, may also be employed for the isolation.

The processes of the present invention is easy to handle, environment friendly, provides better yield with required purity and it may also be practiced at on industrial scale.

Certain specific aspects and embodiments of the present application will be explained in greater detail with reference to the following examples, which are provided only for purposes of illustration and should not be construed as limiting the scope of the application in any manner. Variations of the described procedures, as will be apparent to those skilled in the art, are intended to be within the scope of the present application.

### EXAMPLES

### Example-1: Preparation of 1-(2-fluoro-6-(trifluoromethyl)benzyl)-3-(4-methoxybenzyl)-6-methylpyrimidine-2,4(1H,3H)-dione

1-(2-fluoro-6-(trifluoromethyl)benzyl)-6-methylpyrimidine-2,4(1H,3H)-dione (50 g) and DMF (400 mL) were charged into RBF at 30° C. and stirred for 10 minutes. Potassium carbonate (45.7 g) and 4-Methoxybenzylchloride (31.1 g) were added to the reaction mixture at 30° C. and stirred for 10 minutes. The reaction mixture was heated up to 63° C. and maintained for 4 hours. The progress of the reaction is monitored by HPLC. Upon completion of the reaction, water (1500 mL) was added to the reaction mass and stirred for 10 minutes. Filtered the reaction mass under vacuum and washed with water (250 mL). The obtained solid was dissolved in dichloromethane (100 mL) and washed with water (100 mL). Layers were separated and the organic layer was washed with 5% NaCl solution, followed by dried over on sodium sulphate. The organic layer was concentrated under vacuum at 32° C. to give the title compound. Yield: 91%; Purity by HPLC: 96.32%.

### Example-2: Preparation of 5-bromo-1-(2-fluoro-6-(trifluoromethyl)benzyl)-3-(4-methoxybenzyl)-6-methylpyrimidine-2,4(1H,3H)-dione

1-(2-Fluoro-6-(trifluoromethyl)benzyl)-3-(4-methoxybenzyl)-6-methylpyrimidine-2,4(1H,3H)-dione (90 g), acetonitrile (1350 mL) were charged at 32° C. and cooled to 20° C. N-Bromosuccinimide (41.7 g) was added to the reaction mixture at 20° C. and stirred for 3 hours. The progress of the reaction is monitored by HPLC. Upon completion of the reaction water (1400 mL) was added at 20° C. over a period of one hour. The reaction mixture was stirred for additional one hour. The solid was filtered and washed with water (200 mL). The obtained solid was dried under vacuum at 30° C. to obtain the title compound. Yield: 92 g

### Example-3: Preparation of 5-(2-fluoro-3-methoxyphenyl)-1-(2-fluoro-6-(trifluoromethyl)benzyl)-3-(4-methoxybenzyl)-6-methylpyrimidine-2,4(1H,3H)-dione

5-bromo-1-(2-fluoro-6-(trifluoromethyl)benzyl)-3-(4-methoxybenzyl)-6-methylpyrimidine-2,4(1H,3H)-dione (1 g) dissolved in 1,4-dioxane (30 mL) and 2-fluoro-3-methoxy phenylboronic acid (0.678 g), water (3.5 mL), sodium carbonate (0.843 g) were charged at 30° C. and stirred for 10 minutes. The reaction mixture was heated up to 55° C. 2-Dicyclohexylphosphino-2′,6′-dimethoxybiphenyl/Sphos (0.049 g) and palladium acetate (0.013 g) were added to the reaction mixture at 55° C. and heated up to 72° C., maintained for 3 hours. Filtered the reaction mass through hyflow and washed with ethyl acetate (10 mL). Layers were separated and the aqueous layer was extracted with ethyl acetate (5 mL). Combined the organic layers and the organic layer washed with brine solution. Layers were separated and the organic layer was dried with sodium sulphate. The organic layer was concentrated under vacuum at 45° C. to give the title compound.

### Example-4: Preparation of 5-(2-fluoro-3-methoxyphenyl)-1-(2-fluoro-6-(trifluoromethyl)benzyl)-3-(4-methoxybenzyl)-6-methylpyrimidine-2,4(1H,3H)-dione

5-bromo-1-(2-fluoro-6-(trifluoromethyl)benzyl)-3-(4-methoxybenzyl)-6-methylpyrimidine-2,4(1H,3H)-dione (1 g) dissolved in 1,4-dioxane (30 mL) and 2-fluoro-3-methoxy phenylboronic acid (0.678 g), water (3.5 mL), sodium carbonate (0.843 g) were charged at 30° C. and stirred for 10 minutes. The reaction mixture was heated up to 55° C. 2-Dicyclohexylphosphino-2′,6′-diisopropoxybiphenyl/Ruphos (0.056 g) and palladium acetate (0.013 g) were added to the reaction mixture at 55° C. and heated up to 72° C., maintained for 3 hours. Filtered the reaction mass through hyflow and washed with ethyl acetate (10 mL) and water (10 mL). Layers were separated and the aqueous layer was extracted with ethyl acetate (10 mL). Combined the organic layers and washed with brine solution. Layers were separated and the organic layer was dried with sodium sulphate. The organic layer was concentrated under vacuum at 45° C. to give the title compound.

### Example-5: Preparation of 5-(2-fluoro-3-methoxyphenyl)-1-(2-fluoro-6-(trifluoromethyl) benzyl)-6-methylpyrimidine-2,4(1H,3H)-dione

5-(2-fluoro-3-methoxyphenyl)-1-(2-fluoro-6-(trifluoromethyl)benzyl)-3-(4-methoxybenzyl)-6-methylpyrimidine-2,4(1H,3H)-dione (1.1 g) dissolved in dichloromethane (11 mL) and anisole (1.088 g) were charged at 30° C. and stirred for 10 minutes. The reaction mixture was cooled to 3° C. Aluminiumtrichloride (0.671 g) was slowly added to the reaction mass at 30° C. The reaction mixture was quenched into 10% KOH solution. Ethyl acetate (11 mL) was added to the reaction mass. Both layers were separated. The aqueous layer was extracted with ethyl acetate (11 mL). Layers were separated. Combine the organic layer and washed with brine solution, dried with sodium sulphate. The organic layer was completely distilled under vacuum at 45° C. MTBE (11 mL) was added to the obtained crude and stirred for 10 minutes. Filtered the solid and washed with MTBE (5.5 mL) to give the title compound. Yield: 67%

### Example-6: Preparation of tert-butyl-(R)-(2-(5-(2-fluoro-3-methoxyphenyl)-3-(2-fluoro-6-(trifluoromethyl)benzyl)-4-methyl-2,6-dioxo-3,6-dihydropyrimidin-1(2H)-yl)-1-phenylethyl)carbamate

5-(2-fluoro-3-methoxyphenyl)-1-(2-fluoro-6-(trifluoromethyl)benzyl)-6-methylpyrimidine-2,4(1H,3H)-dione (10 g), THF (120 mL) were charged at 27° C. and stirred for 10 minutes. Tert-butyl-(R)-(2-hydroxy-1-phenylethyl) carbamate (6.12 g), triphenylphosphine (10.46 g) and di-tert-butyl (E)-diazene-1,2-dicarboxylate (9.18 g) were charged in to the reaction mixture at 27° C. and stirred for 10 minutes. The reaction mass was heated up to 42° C. and maintained for 5 hours. The solvent from reaction mass was evaporated at 28° C. MTBE (30 mL) was added to the obtained crude and stirred for 10 minutes. Filtered the solid under vacuum and washed with MTBE (10 mL). The obtained filtrate was completely evaporated under vacuum. Toluene (20 mL) was added to the obtained crude and stirred for 10 minutes. Magnesium chloride (6.70 g) was added to the reaction mass and stirred for 10 minutes. The reaction mass was heated up to 65° C. and maintained for two hours. Filtered the reaction mass and washed with toluene (20 mL). The obtained filtrate was concentrated under vacuum at 45° C. to give the title compound.

### Example-7: Preparation of (R)-3-(2-amino-2-phenylethyl)-5-(2-fluoro-3-methoxy phenyl)-1-(2-fluoro-6-(trifluoromethyl)benzyl)-6-methylpyrimidine-2,4(1H,3H)-dione Oxalate Salt

Tert-butyl(R)-(2-(5-(2-fluoro-3-methoxyphenyl)-3-(2-fluoro-6-(trifluoromethyl)benzyl)-4-methyl-2,6-dioxo-3,6-dihydropyrimidin-1(2H)-yl)-1-phenylethyl)carbamate (5 g) was dissolved in dichloromethane (25 mL) at 30° C. The reaction mixture was cooled to 4° C. Trifluoro acetic acid (5.30 g) was added to the reaction mass at 4° C. and maintained for 60 minutes. Trifluoro acetic acid (0.883 g) was slowly added to the reaction at 30° C. and maintained for 4 hours. The reaction mass was cooled to 12° C. Sodium bicarbonate solution (50 mL) was added to the reaction mass at 12° C. and stirred for 5 minutes. Layers were separated and the organic layer was washed with sodium bicarbonate (50 mL) and water (20 mL). The solvent form the organic layer was concentrated under vacuum at 30° C. MTBE (20 mL) was added to the obtained crude at 30° C. and stirred for 10 minutes. Oxalic acid (0.906 g) was added to the reaction mass at 30° C. and maintained for 90 minutes. Decant the solvent from the reaction mass and MTBE (20 mL) was added to the reaction mass at 30° C., stirred for 10 minutes. Filtered the solid and washed with MTBE (10 mL), dried under vacuum at 30° C. to give the title compound.

### Example-8: Preparation of (R)-3-(2-amino-2-phenylethyl)-5-(2-fluoro-3-methoxy phenyl)-1-(2-fluoro-6-(trifluoromethyl)benzyl)-6-methylpyrimidine-2,4(1H,3H)-dione Hydrochloride Salt

Tert-butyl(R)-(2-(5-(2-fluoro-3-methoxyphenyl)-3-(2-fluoro-6-(trifluoromethyl)benzyl)-4-methyl-2,6-dioxo-3,6-dihydropyrimidin-1(2H)-yl)-1-phenylethyl)carbamate (5 g) was dissolved in dichloromethane (25 mL) at 30° C. The reaction mixture was cooled to 4° C. Trifluoro acetic acid (5.30 g) was added to the reaction mass at 4° C. and maintained for 60 minutes. Trifluoro acetic acid (0.883 g) was slowly added to the reaction at 30° C. and maintained for 4 hours. The reaction mass was cooled to 12° C. Sodium bicarbonate solution (50 mL) was added to the reaction mass at 12° C. and stirred for 5 minutes. Layers were separated and the organic layer was washed with sodium bicarbonate (50 mL) and water (20 mL). The solvent form the organic layer was concentrated under vacuum at 30° C. MTBE (20 mL) was added to the obtained crude at 30° C. and stirred for 10 minutes. The reaction mixture was cooled to 12° C. Hydrochloric acid was purged to the reaction mass at 12° C. and maintained for 60 minutes. Filtered the solid and washed with MTBE (10 mL), dried under vacuum at 32° C. to give the title compound.

### Example-9: Preparation of (R)-3-(2-amino-2-phenylethyl)-5-(2-fluoro-3-methoxyphenyl)-1-(2-fluoro-6-(trifluoromethyl)benzyl)-6-methylpyrimidine-2,4(1H,3H)-dione

Tert-butyl-(R)-(2-(5-(2-fluoro-3-methoxyphenyl)-3-(2-fluoro-6-(trifluoromethyl) benzyl)-4-methyl-2,6-dioxo-3,6-dihydropyrimidin-1(2H)-yl)-1-phenylethyl)carbamate (20 g) dissolved in dichloromethane (100 mL). The reaction mixture was cooled to 5° C. Trifluoro acetic acid (21.19 g) was slowly added to the reaction mass at 6° C. The reaction mixture temperature was raised to 25° C. and maintained for 14 hours. The reaction mass was cooled to 10° C. Water (50 mL) and sodium bicarbonate (100 mL) were added to the reaction mass at 10° C. and stirred for 10 minutes. Both layers were separated and the aqueous layer was extracted with dichloromethane (60 mL). Combined the both organic layers and washed with water (60 mL). The solvent from the organic layer was completely evaporated under vacuum. MTBE (150 mL) was added to the obtained crude and stirred for 10 minutes. Oxalic acid dihydrate (2.54 g) was added to reaction mixture at 29° C. and maintained for two hours. The obtained solid was filtered. MTBE (40 mL) was added to the filtrate at 29° C. and maintained for 30 minutes. Filtered the obtained solid at 29° C. To the combine solids Ethyl acetate was added. 5% aqueous potassium hydroxide (100 mL) was added to the reaction mass and stirred for 10 minutes. Both layers were separated. The aqueous layer was extracted with ethyl acetate. Combine the both organic layers and washed with water, dried with sodium sulphate. The solvent from organic layer was concentrated under vacuum at 48° C. to give the title compound.

### Example-10: Preparation of ethyl (R)-4-((2-(5-(2-fluoro-3-methoxyphenyl)-3-(2-fluoro-6-(trifluoromethyl)benzyl)-4-methyl-2,6-dioxo-3,6-dihydropyrimidin-1(2H)-yl)-1-phenylethyl)amino)butanoate

(R)-3-(2-amino-2-phenylethyl)-5-(2-fluoro-3-methoxyphenyl)-1-(2-fluoro-6-(trifluoromethyl)benzyl)-6-methylpyrimidine-2,4(1H,3H)-dione (1.4 g) dissolved in DMF (7 mL), DIPEA (0.763 g) and ethyl 4-bromobutanoate (0.501 g) were charged at 28° C. The reaction mixture was heated up to 68° C. and maintained for 6 hours. The reaction mass was quenched with cold water (30 mL). The aqueous layer was extracted with MTBE (2×28 mL). Combine the organic layer and washed with water (30 mL), dried with sodium sulphate. The solvent form the organic layer was concentrated under vacuum at 45° C. to give the title compound. Yield: 82.6%

### Example-11: Preparation of Elagolix Sodium (I)

Ethyl (R)-4-((2-(5-(2-fluoro-3-methoxyphenyl)-3-(2-fluoro-6-(trifluoromethyl)benzyl)-4-methyl-2,6-dioxo-3,6-dihydropyrimidin-1(2H)-yl)-1-phenylethyl)amino)butanoate (16.5 g) dissolved in ethanol (83 mL) and stirred for 10 minutes. Sodium hydroxide solution was slowly added to the reaction mass at 31° C. and maintained for 6 hours. The solvent from the reaction mass was evaporated completely at 31° C. Water (66 mL) and MIBK (100 mL) were added to the obtained crude and stirred for 10 minutes. The reaction mass was heated to 55° C. Layers were separated. MIBK (100 mL) was added to the aqueous layer. The reaction mass was heated to 55° C. and the layers were separated. NaOH solution (25 mL) was added to the aqueous layer and stirred for 10 minutes. MIBK (100 mL) was added to the aqueous layers and layers were separated. The aqueous layer was extracted with MIBK (100 mL). Combine the organic layer and the solvent from the organic layers was evaporated under vacuum at 45° C. MIBK (15 mL) was added to the obtained crude and stirred for 10 minutes. The reaction mixture was slowly added to the heptane (200 mL) and stirred for 30 minutes. Filtered the solid and washed with heptane (20 mL) to give the title compound. Yield: 60%

### Example-12: Preparation of tert-butyl (R)-(2-(5-(2-fluoro-3-methoxyphenyl)-3-(2-fluoro-6-(trifluoromethyl)benzyl)-4-methyl-2,6-dioxo-3,6-dihydropyrimidin-1(2H)-yl)-1-phenylethyl)carbamate

Tert-butyl (R)-(2-(5-bromo-3-(2-fluoro-6-(trifluoromethyl)benzyl)-4-methyl-2,6-dioxo-3,6-dihydropyrimidin-1(2H)-yl)-1-phenylethyl)carbamate (1.0 g), 3H)-dione (1 g) dissolved in 1,4-dioxane (30 mL) and 2-fluoro-3-methoxy phenylboronic acid (0.566 g), water (3.5 mL), sodium carbonate (0.706 g) were charged at 30° C. and stirred for 10 minutes. The reaction mixture was heated up to 55° C. 2-Dicyclohexylphosphino-2′,6′-diisopropoxybiphenyl/Ruphos (0.016 g) and palladium acetate (3.74 mg) were added to the reaction mixture at 56° C. and heated up to 72° C., maintained for 3 hours. Filtered the reaction mass through hyflow and washed with MTBE (20 mL). Layers were separated and the organic layer was washed with water (20 mL). Layers were separated and the organic layer was washed with brine solution. The organic layer was concentrated under vacuum at 45° C. to give the title compound.

### Example-13: Preparation of 5-(2-fluoro-3-methoxyphenyl)-1-(2-fluoro-6-(trifluoromethyl)benzyl)-6-methylpyrimidine-2,4(1H,3H)-dione

5-bromo-1-(2-fluoro-6-(trifluoromethyl)benzyl)-6-methylpyrimidine-2,4(1H,3H)-dione (1 g), acetone (5 mL) and water (5 mL) were charged at 30° C. 2-fluoro-3-methoxy phenylboronic acid (0.669 g), potassium hydroxide (0.442 g) were charged at 30° C. and stirred for 10 minutes. 2-Dicyclohexylphosphino-2′,6′-diisopropoxybiphenyl (Ruphos) (0.245 g) and palladium acetate (0.059 g) were added to the reaction mixture at 30° C. The reaction mass was heated to 52° C. and maintained for 6 hours. The solvent from the reaction mass was evaporated under vacuum at 52° C. to give the title compound.

### Example-14: Preparation of 1-(2-fluoro-6-(trifluoromethyl)benzyl)-3-(4-methoxybenzyl)-6-methylpyrimidine-2,4(1H,3H)-dione

1-(2-fluoro-6-(trifluoromethyl)benzyl)-6-methylpyrimidine-2,4(1H,3H)-dione (100 g) and DMF (800 mL) were charged into RBF at 30° C. and stirred for 10 minutes. Potassium carbonate (114 g) and 4-Methoxybenzylchloride (62.2 g) were added to the reaction mixture at 30° C. and stirred for 10 minutes. The reaction mixture was heated up to 63° C. and maintained for 4 hours. The progress of the reaction is monitored by HPLC. Filtered the reaction mass through hyflow and washed with DMF (100 mL). The obtained filtrate was taken into RBF and cooled to 15-20° C. Water (900 mL) was added to the reaction mass and stirred for 10 minutes. Filtered the reaction mass under vacuum and washed with water (2×200 mL), dried under vacuum at 50° C. to give the title compound. Yield: 79.3%; Purity by HPLC: 97.06%

### Example-15: Preparation of 5-bromo-1-(2-fluoro-6-(trifluoromethyl)benzyl)-3-(4-methoxybenzyl)-6-methylpyrimidine-2,4(1H,3H)-dione

1-(2-Fluoro-6-(trifluoromethyl)benzyl)-3-(4-methoxybenzyl)-6-methylpyrimidine-2,4(1H,3H)-dione (70 g), acetonitrile (910 mL) were charged at 32° C. and cooled to 15° C. N-Bromosuccinimide (30.7 g) was added to the reaction mixture at 20° C. and washed with acetonitrile (70 mL), maintained for 3-5 hours. The progress of the reaction is monitored by HPLC. Upon completion of the reaction water (1050 mL) was added at 16° C. over a period of one hour. The reaction mixture was stirred for additional one hour. The solid was filtered and washed with water (2×140 mL). The obtained solid was dried under vacuum at 50° C. to obtain the title compound.

Purity by HPLC: 96.87%

### Example-16: Preparation of 5-(2-fluoro-3-methoxyphenyl)-1-(2-fluoro-6-(trifluoromethyl) benzyl)-6-methylpyrimidine-2,4(1H,3H)-dione

5-bromo-1-(2-fluoro-6-(trifluoromethyl)benzyl)-3-(4-methoxybenzyl)-6-methyl pyrimidine-2,4(1H,3H)-dione (25 g) dissolved in 1,4-dioxane (350 mL) and 2-fluoro-3-methoxy phenylboronic acid (14.83 g), water (50 mL), sodium carbonate (0.843 g) were charged at 30° C. and stirred for 10 minutes. The reaction mixture was heated up to 71° C. Ru-phos (0.186 g) and palladium acetate (0.045 g) were added to the reaction mixture at 71° C. and degassed with 1,4-dioxane (25 mL) for 30 minutes and heated up to 72° C., maintained for 3 hours. The reaction mass cooled to 30° C. and charged toluene (125 mL) and water (250 mL) to the reaction mass, stirred for 10 minutes. Filtered the reaction mass through hyflow and washed with toluene (2×125 mL). Layers were separated and the organic layer was washed with water (2×125 mL). Combined the organic layers and the organic layer washed with sodium chloride solution (12.5 g NaCl in 12 mL water). The organic layer was concentrated under vacuum at 45° C. up to 2-3 volume. Toluene (125 mL) was added to the reaction mass. Again the organic layer was concentrated under vacuum at 45° C. up to 2-3 volume. Toluene (250 mL) was added to the reaction mass and stirred for 10 minutes. Anisole (18.88 g) and triflic acid (13.10 g) were added to the reaction mass at 30° C. The reaction mixture was heated to 60-65° C. and maintained for 6-8 hours. The reaction mass was cooled to 8° C. DIPEA (17.73 g) was added to the reaction mass at 9° C. and stirred for 5-10 minutes. Heptane (300 mL) was added to the reaction mass at 25° C. and stirred for 5 to 10 minutes. The reaction mass was heated to 52° C. and maintained for 2-3 hours. The reaction mass was cooled to 30° C. and maintained for 30-60 minutes. Filtered the reaction mass and washed with heptane (125 mL) to get the compound.

The obtained wet compound, water (250 mL) were charged at 30° C. and stirred for 30 minutes. Filtered the reaction mass and washed with water (125 mL) to get the compound. The obtained wet compound, acetone (250 mL), potassium carbonate (0.345 g) were charged at 30° C. and stirred for 5-10 minutes. The reaction mixture was heated to 42° C. and maintained for 1-2 hours. The reaction mass was cooled to 30° C. Water (200 mL) was added to the reaction mass and stirred for 1-2 hours. Filtered the reaction mass and washed with water (250 mL), dried under vacuum at 55° C. to give the title compound.

Yield: 79.02%; Purity by HPLC: 99.51%

### Example-17: Preparation of tert-butyl-(R)-(2-(5-(2-fluoro-3-methoxyphenyl)-3-(2-fluoro-6-(trifluoromethyl)benzyl)-4-methyl-2,6-dioxo-3,6-dihydropyrimidin-1(2H)-yl)-1-phenylethyl)carbamate

5-(2-fluoro-3-methoxyphenyl)-1-(2-fluoro-6-(trifluoromethyl)benzyl)-6-methyl pyrimidine-2,4(1H,3H)-dione (100 g), DMF (1200 mL), potassium carbonate (162 g) were charged at 28° C. and stirred for 10 minutes. (R)-2-((tert-butoxycarbonyl)amino)-2-phenylethyl methanesulfonate (185 g) was charged in to the reaction mixture at 28° C. and stirred for 10 minutes. The reaction mass was heated up to 50° C. and maintained for 5-6 hours. Isopropyl acetate (500 mL), DM-water (2000 mL) were charged to the reaction mass at 30° C. Both layers were separated. Isopropyl acetate (300 mL) was added to the aqueous layer and stirred for 15 minutes. Combine the organic layers. 10% NaCl solution was added to the organic layer and stirred for 20-30 minutes. Both layers were separated and the organic layer carried forward to the next step.

### Example-18: Preparation of (R)-3-(2-amino-2-phenylethyl)-5-(2-fluoro-3-methoxy phenyl)-1-(2-fluoro-6-(trifluoromethyl)benzyl)-6-methylpyrimidine-2,4(1H,3H)-dione

The obtained organic layer from example-17 charged at RBF at 28° C. and cooled to 20° C. Methane sulfonic acid (50.7 g) was slowly added to the reaction mixture at 20° C. The reaction mass was heated to 52° C. and maintained for 6-8 hours. Aqueous potassium carbonate solution (100 g in 1000 ml water) was added to the reaction mass at 23° C. and stirred for 10 minutes. Both layers were separated. Aqueous H3PO4 solution (100 g in 1000 mL) water was added to the organic layer and stirred for 20 minutes. Both layers were separated. Isopropyl acetate (2×500 mL) was added to the aqueous layer and stirred for 20 minutes. Isopropyl acetate (500 mL) was added to the aqueous layer and stirred for 20 minutes. Aqueous potassium carbonate solution (100 g in 1000 ml water) was added to the reaction mass at 23° C. and stirred for 10 minutes. Both layers were separated. Extracted the aqueous layer with isopropyl acetate (300 mL). Combine the organic layers and washed with water (500 mL). Both layers were separated. The organic layer was washed with 1% NaCl solution and layers were separated. The organic layer was washed with 10% NaCl solution. The solvent from the organic layer was concentrated under vacuum at 43° C. The obtained crude was dissolved in DMF (100 mL) and carried forward to the next step.

Yield: 85%; Purity: 93.2%

### Example-19: Preparation of ethyl (R)-4-((2-(5-(2-fluoro-3-methoxyphenyl)-3-(2-fluoro-6-(trifluoromethyl)benzyl)-4-methyl-2,6-dioxo-3,6-dihydropyrimidin-1(2H)-yl)-1-phenylethyl)amino)butanoate

(R)-3-(2-amino-2-phenylethyl)-5-(2-fluoro-3-methoxyphenyl)-1-(2-fluoro-6-(trifluoromethyl)benzyl)-6-methylpyrimidine-2,4(1H,3H)-dione (100 g) dissolved in DMF (180 mL), DIPEA (45.6 g) and ethyl 4-bromobutanoate (47.9 g) were charged at 28° C. The reaction mixture was heated up to 52° C. and maintained for 5-6 hours. Tert-butyl acetate (600 mL), Water (500 mL) were added to the reaction mass and stirred for 20 minutes. Both layers were separated. Water (500 mL) was added to the organic layer and stirred for 20 minutes. Both layers were separated, 5% aqueous H3PO4 solution (2000 mL) was added to the organic layer and stirred for 20 minutes. Both layers were separated, 5% aqueous H3PO4 solution (500 mL) was added to the organic layer and stirred for 20 minutes. Both layers were separated. Combine the aqueous layers are in RBF at 27° C. Tert-butyl acetate (2×150 mL) was added to the aqueous layer and stirred for 15 minutes. Both layers were separated. Combine the aqueous layers are in RBF at 27° C. dichloromethane (1000 mL) was added to the aqueous layer and stirred for 15 minutes. 10% aqueous potassium carbonate solution was added to the reaction mixture and stirred for 20 minutes. Both layers were separated and the solvent from the organic layer was concentrated under vacuum at 40° C., followed by silica gel bed in column using silica gel 100-200 mesh and dichloromethane (300 mL) to get the title compound Purity by HPLC: 93.84%

### Example-20: Preparation of Elagolix Sodium (I)

Ethyl (R)-4-((2-(5-(2-fluoro-3-methoxyphenyl)-3-(2-fluoro-6-(trifluoromethyl)benzyl)-4-methyl-2,6-dioxo-3,6-dihydropyrimidin-1(2H)-yl)-1-phenylethyl)amino)butanoate (102.7 g) dissolved in ethanol (565 mL) and stirred for 10 minutes. Sodium hydroxide solution was slowly added to the reaction mass at 31° C. and maintained for 6 hours. The solvent from the reaction mass was evaporated completely at 31° C. Water (1027 mL) and MIBK (719 mL) were added to the obtained crude and stirred for 10 minutes. The reaction mass was heated to 55° C. and maintained for 20 minutes. Layers were separated. MIBK (2×719 mL) was added to the aqueous layer. The reaction mass was heated to 55° C. and the layers were separated. NaOH (7.53 g), sodium chloride, MIBK (719 mL) were was added to the aqueous layer and stirred for 20 minutes. Both layers were separated and the aqueous layer was washed with sodium chloride solution. The solvent from organic layer was concentrated up to 2-3 vol under vacuum at 40° C. MIBK (2×205 mL) was added to the obtained solution and evaporated under vacuum. Filtered the reaction mass through hyflow and washed with MIBK (51.4 mL) to get the MIBK layer. n-Heptane (1284 mL) and MTBE (1284 ML) are charged in another RBF and stirred for 10 minutes. MIBK layer was slowly added to the reaction mixture and stirred for 1-2 hours. Filtered the obtained solid and washed with n-heptane (2×514 mL) to give the title compound.

Yield: 56.9%; Purity by HPLC: 99.48%

1. An improved process for the preparation of Elagolix sodium (I) which includes one or more of the following steps, individually or in the sequence recited:
a) N-protection of compound of formula (IV) to obtain compound of formula (V), 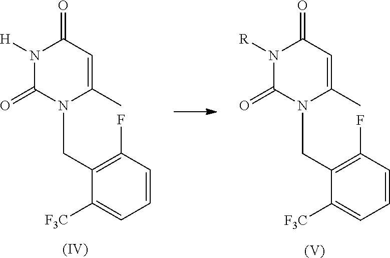
wherein R is para methoxy benzyl, tert-butyloxycarbonyl, methoxymethyl and fluorenylmethyloxycarbonyl;
b) bromination of compound of formula (V) with a brominating agent to obtain a compound of formula (VI), 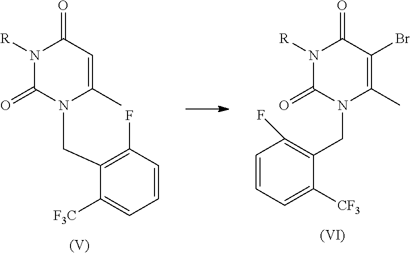
wherein R is para methoxy benzyl, tert-butyloxycarbonyl, methoxy methyl and fluorenylmethyloxycarbonyl;
c) reacting a compound of formula (VI) with (2-fluoro-3-methoxyphenyl)boronic acid to obtain a compound of formula (VII), 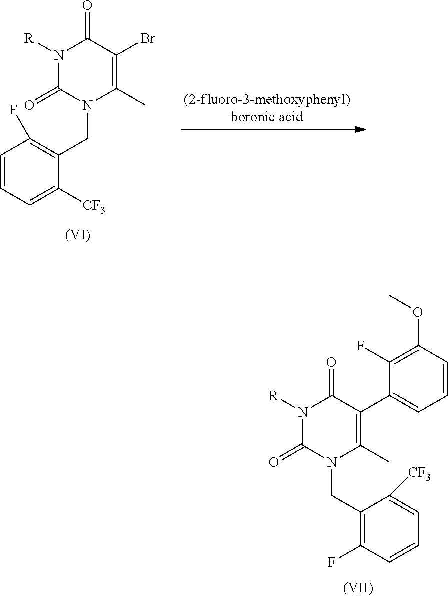
wherein R is para methoxy benzyl, tert-butyloxycarbonyl, methoxymethyl and fluorenylmethyloxycarbonyl;
d) reacting a compound of formula (VII) with a reagent to obtain a compound of formula (VII), 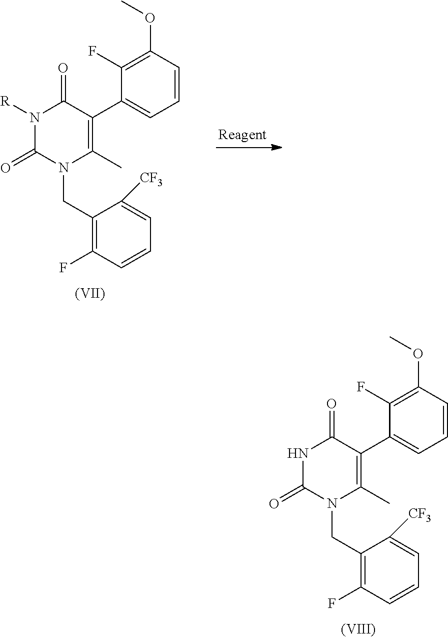
wherein R is para methoxy benzyl, tert-butyloxycarbonyl, methoxymethyl and fluorenylmethyloxycarbonyl;
e) reacting a compound of formula (VIII) with (R)-2-((tert-butoxycarbonyl)amino)-2-phenylethyl methanesulfonate (or) tert-butyl (R)-(2-hydroxy-1-phenylethyl) carbamate to obtain a compound of formula (IX); 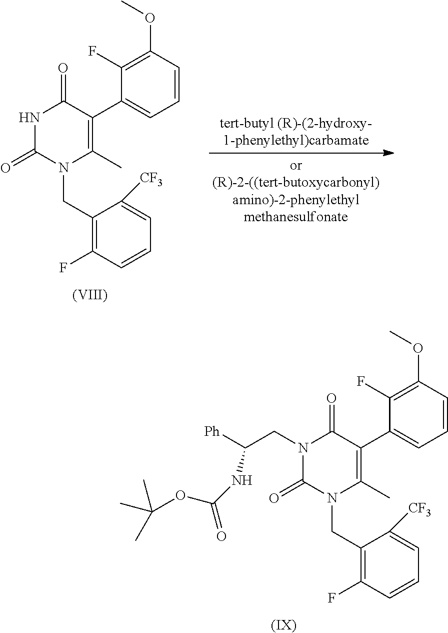
f) converting a compound of formula (IX) in to a compound of formula (XI) or its pharmaceutically acceptable salts; 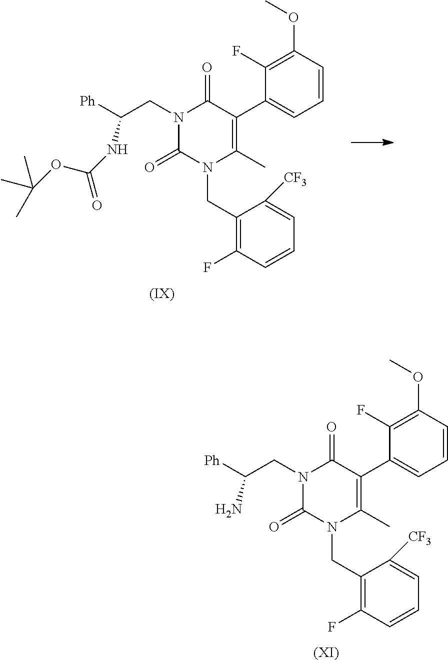
g) reacting a compound of formula (XI) with ethyl 4-bromobutanoate to obtain ethyl ester of Elagolix of formula (XII); and 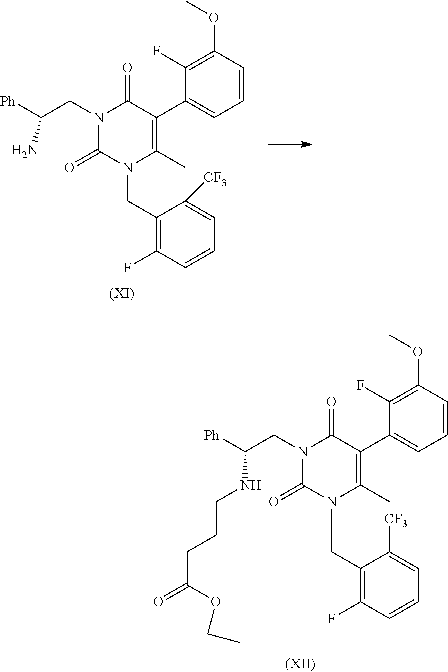
h) converting ethyl ester of Elagolix of formula (XII) into Elagolix sodium of formula (I).

2) (canceled)

3) The process according to claim 1, wherein brominating agent in step b) is selected from phosphorus tribromide, aluminum tribromide, N-bromosuccinimide (NBS), liquid bromine, N-bromoacetamide, N-bromophthalimide, N-bromosaccharin, benzyltrimethylammoniumTribromide, TrimethylphenylammoniumTribromide, and 1,3-Dibromo-5,5-Dimethylhydantoin (DBDMH).

4) The process according to claim 1, wherein palladium reagent in step c) is selected from palladium acetate, palladium(II) chloride, palladium(II) bromide, palladium(II) iodide, palladium(II) nitrate tris(dibenzylideneacetone)dipalladium(0), 1,1′-bis(di-tert-butyl phosphino ferrocene) PdC12 and tetrakis triphenylphosphine palladium.

5) The process according to claim 1, wherein ligand in step c) is selected from 2-dicyclohexylphosphino-2′,6′-diisopropoxybiphenyl (Ruphos), 2-dicyclohexylphosphino-2′,6′-dimethoxybiphenyl (Sphos), 2-dicyclohexylphosphino-2′-methylbiphenyl, 2-methyl-2′-dicyclohexylphosphinobiphenyl (Mephos), 2-dicyclohexylphosphino-2′-(N,N-dimethylamino)biphenyl (davephos), (2-Biphenyl)di-tert-butylphosphine, (2-Biphenylyl)di-tert-butylphosphine, 2-(Di-tert-butylphosphine) biphenyl (Johnphos), 2-dicyclohexylphosphino-2′,4′,6′-triisopropylbiphenyl (Xphos), and 4,5-Bis(diphenylphosphino)-9,9-dimethylxanthene (Xantphos).

6) The process according to claim 1, wherein base used in step c) and step e) is selected from potassium carbonate, sodium carbonate, cesium carbonate, lithium carbonate, sodium bicarbonate, potassium bicarbonate, potassium hydrogen phosphate, sodium hydroxide, potassium hydroxide, barium hydroxide, potassium fluoride, tetrabutyl ammonium fluoride, triethylamine, tributylamine, N-Methyl-2-pyrrolidone (NMP), N-methyl morpholine, DBU, and DABCO.

7) The process according to claim 1, wherein acid used in step d) is selected from aluminum, trichloride, acetic acid, ceric ammonium nitrate, hydrochloride, hydrobromide, trifluoro acetic acid, and triflic acid.

8) The process according to claim 1, wherein acid used in step f) is selected from hydrochloric acid, sulphuric acid, hydrobromic acid, acetic acid, orthophosphoric acid, trifluoro acetic acid, and methane sulfonic acid.

9) Compounds of formula (V), (VI) and (X). 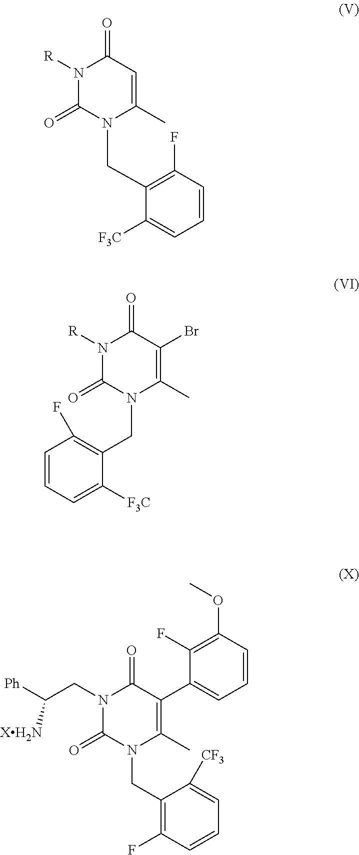
wherein R is para methoxy benzyl, tert-butyloxycarbonyl, methoxymethyl and fluorenylmethyloxycarbonyl; and
wherein X in compound of formula (X) is hydrochloride, oxalic acid, hydrobromide, tartaric acid, mandelic acid, dibenzoyl-L-tartaric acid and malic acid.

10) Compounds of formula (Xa) and (Xb).

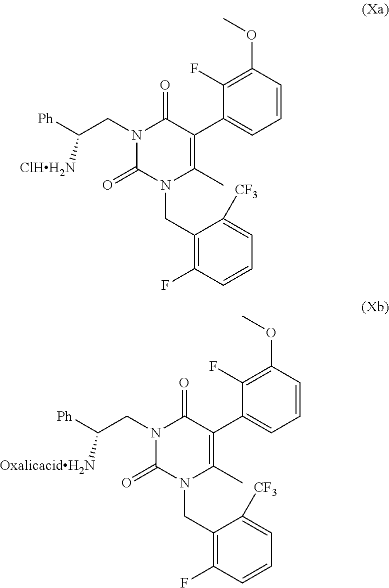

11) The compounds of formula (V), (VI), and (X), of claim 9, for use in the preparation of Elagolix sodium of formula (I).

12) The compounds of formula (Xa) and (Xb) of claim 10, for use in the preparation of Elagolix sodium of formula (I).
